(19) United States
(12) Plant Patent Application Publication
(10) Pub. No.: US 2005/0262604 P1
(43) Pub. Date: Nov. 24, 2005

(54) Buxus sinica var. insularis - 'WEE WILLIE'

(50) BUXUX SINICA; BOXWOOD

(76) Inventor: William E. Schubert, Austinburg, OH (US)

Correspondence Address: WAYNE L. LOVERCHECK, THE QUINN LAW FIRM, 2222 W. GRANDVIEW BLVD., ERIE, PA, 16506-4508, US

(21) Appl. No.: 10/848,630

(22) Filed: May 20, 2004

(51) Int. Cl. A01H 5/00

(52) U.S. Cl. PLT/226

(57) ABSTRACT

A new and distinct cultivar of boxwood that originated as a branch sport. The orderly arrangement of leaves on each stem gives this shrub considerable textural interest. This shrub displays excellent hardiness, and its emerald-green leaves retain their color through the winter. This semi-dwarf evergreen shrub displays an upright, dense growth habit, making it suitable for use in small gardens or as a low, formal hedge or border.

### CROSS REFERENCE TO RELATED APPLICATIONS

None

### STATEMENT REGARDING FEDERALLY SPONSORED RESEARCH OR DEVELOPMENT

Not Applicable

### BRIEF SUMMARY OF THE INVENTION

The invention related to a new and distinct cultivar of boxwood, Buxus sinica var. insularis (synonym: Buxus microphylla var. koreana) that originated as a branch sport on the variety and was first observed in 1990, at Tree Tyme Nursery, Inc. in Austinburg, Ohio. Rooted cuttings have been propagated by the inventor each year since 1990. The cultivar is distinguished by its decussate leaf arrangement, with successive pairs of leaves rotated 90 degrees on the stem axis from the previous pair. In this arrangement, its foliage resembles that of the box-leaved hebe, Hebe buxifolia, but we know of no other boxwood that displays this orderly leaf arrangement. Combined with the vertical orientation of the stems, the leaf arrangement gives a unique and attractive texture to this shrub.

The new cultivar is a dense-growing semi-dwarf broadleaved evergreen, reaching a height and spread of 24 inches by 24 inches in six years from a rooted cutting. This cultivar is propagated from rooted cuttings, and plants propagated by this method consistently and uniformly display the unique characteristics of the original plant.

### BRIEF DESCRIPTION OF THE PHOTOGRAPHS

FIG. 1 shows propagation flat of rooted cuttings. Emerald green leaves with upright growth habit displaying pubescent stems. 1 year.

FIG. 2 shows propagation flat of rooted cuttings showing decussate arrangement of leaves on stems. 1 year.

FIG. 3 shows rooted cutting in a 1×1 cm pot noting upright growth with dense compact habit. 2 year.

FIG. 4 shows rooted cutting in 1×1 cm pot showing its successive pairs of leaves rotated 90 degrees on the stem axis from the previous pair. 2 year 4″

FIG. 5 shows rooted cutting displaying the consistent uniformity of the original plant. 2 year.

FIG. 6 shows rooted cutting displaying the consistent uniformity of the original plant. 2 year.

FIG. 7 shows finished material highlighting the rich emerald green color against its pubescent stems. 3 year 10″×12″

FIG. 8 shows finished material highlighting the rich emerald green color against its pubescent stems. 3 year 10″×12″

FIG. 9 shows finished material highlighting the rich emerald green color against its pubescent stems. 3 year 10″×12″

FIG. 10 shows specimen material. Extraordinary density and texture makes this shrub an excellent choice for small landscape or low formal hedge. 4 year 12″×15″

FIG. 11 shows specimen material. Extraordinary density and texture makes this shrub an excellent choice for small landscape or low formal hedge. 4 year 12″×15″

FIG. 12 shows specimen material. Extraordinary density and texture makes this shrub an excellent choice for small landscape or low formal hedge. 4 year 12″×15″

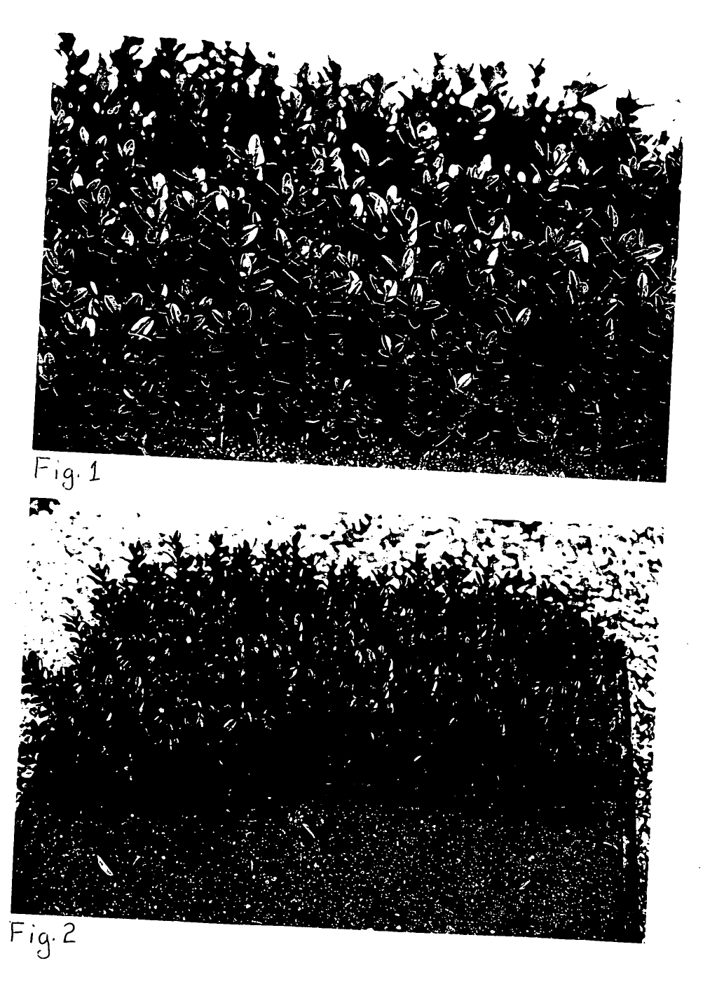

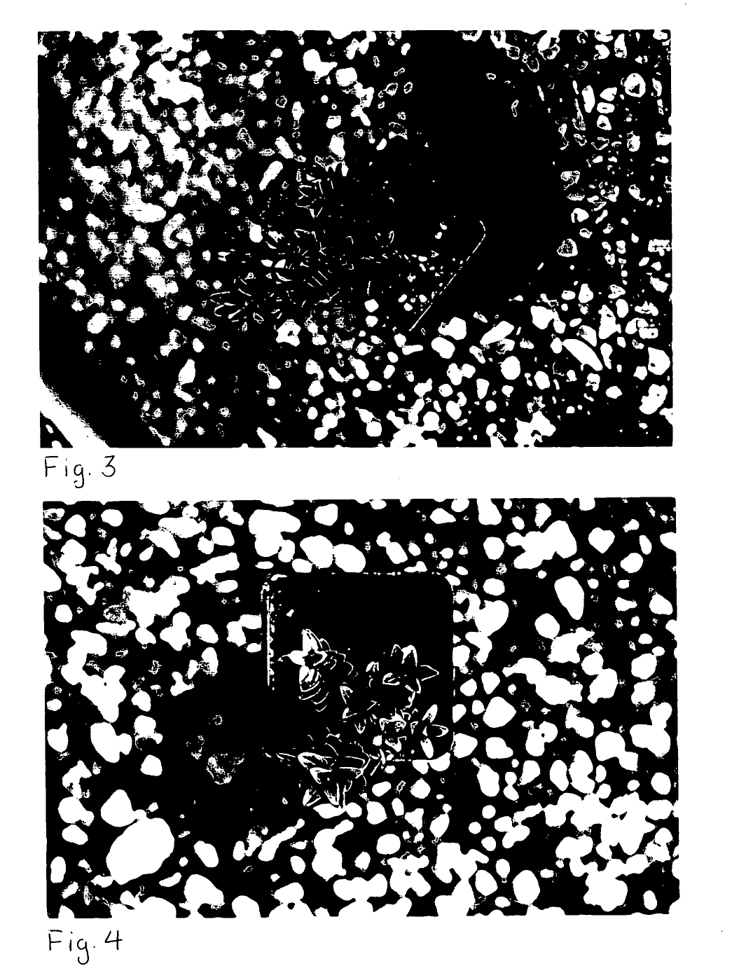

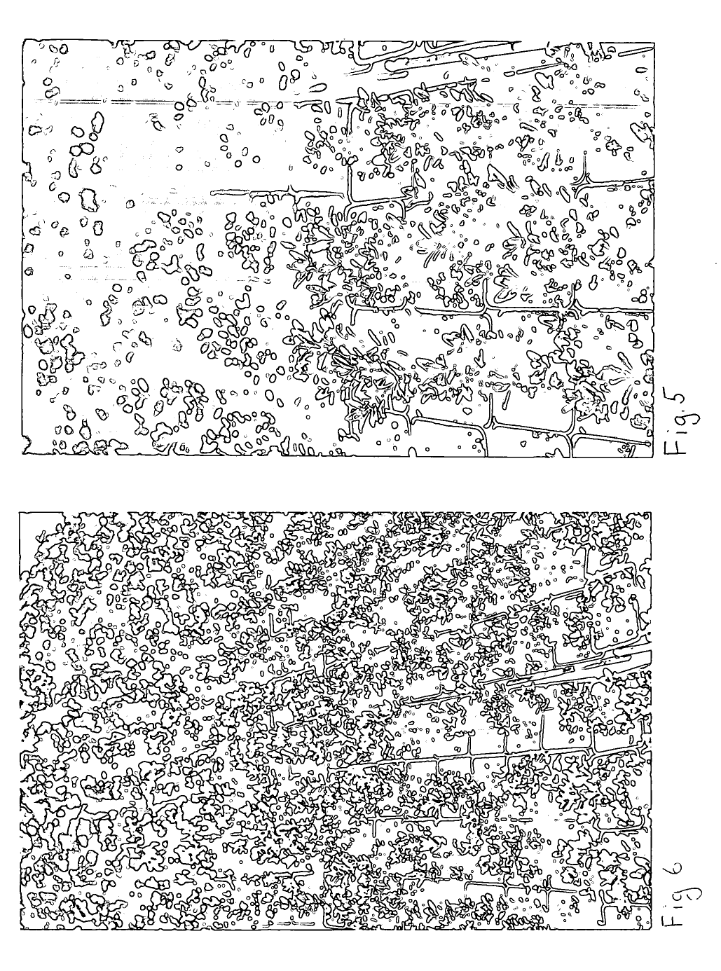

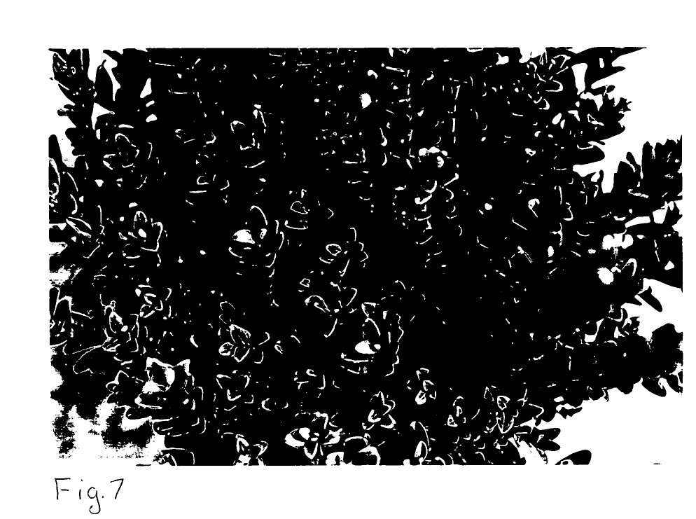

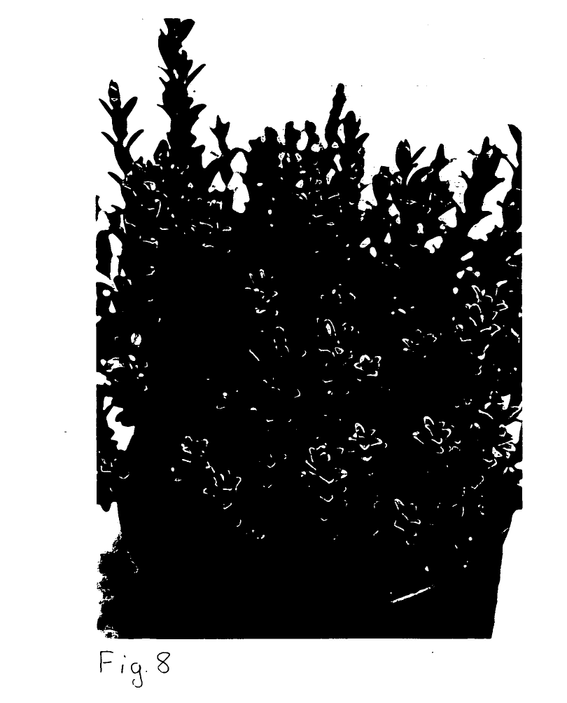

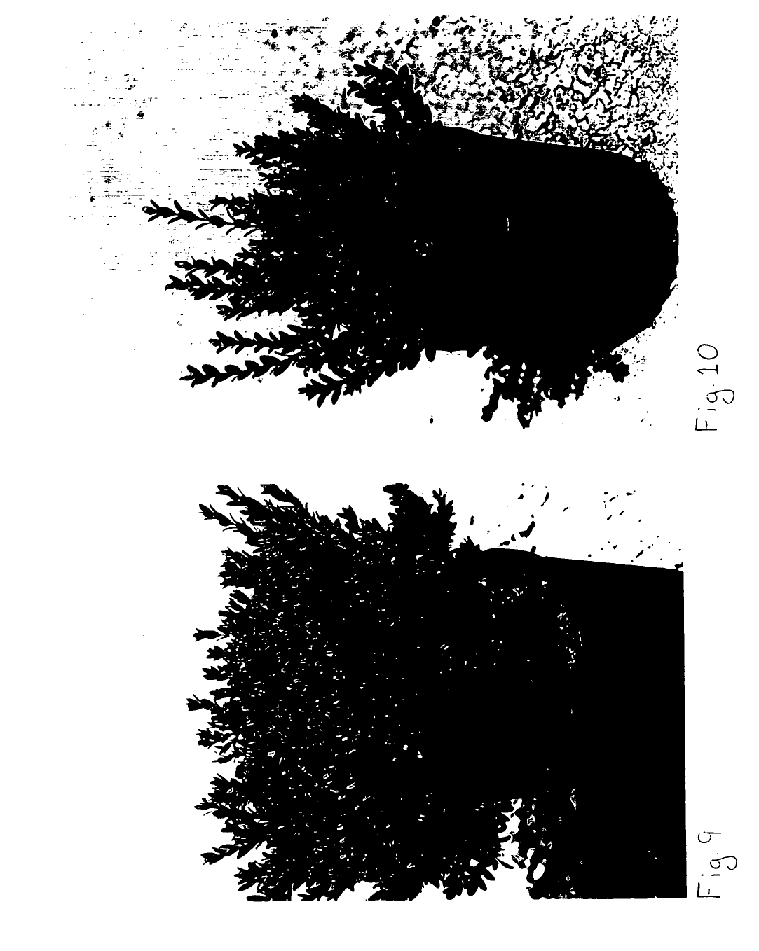

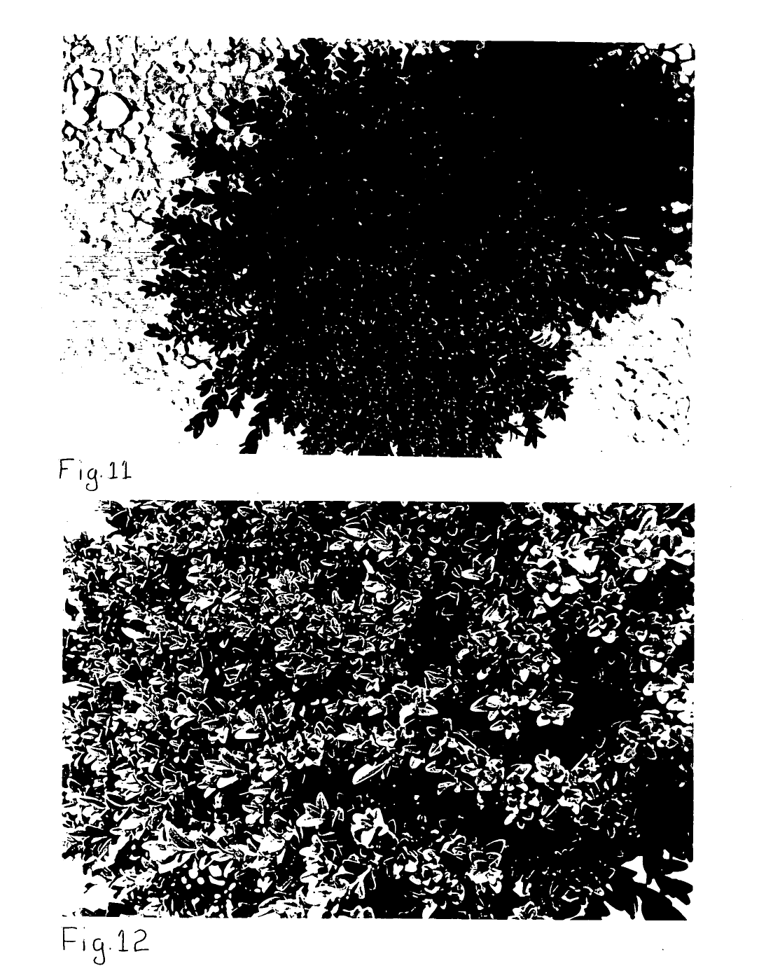

### DETAILED BOTANICAL DESCRIPTION

Parentage: originated prior to 1990 as a branch sport on a specimen of Buxus sinica var. insularis.
Form/size: Compact shrub with upright branches, to two feet in height by two feet in spread in six years. Mature dimensions are expected to be consistent.
Hardiness: Fully hardy in USDA hardiness Zone 5. Probable hardiness in Zone 4. Has withstood minimum temperature of −20° F. without injury.
Growth rate: Slow, average of four inches per year.
Stems: Medium green the first year, turning to light tan in the second year, as for the species. Young stems are pubescent.
Leaves: Evergreen, lanceolate-elliptic with rounded apices and acute bases. Leaves borne in decussate arrangement on the stem. Leaf blades 1.5-2 cm. Long and 0.7-1.0 cm wide at the widest point. Leaf stalks 0.1-0.2 cm in length. Sparse pubescence on midrib and leaf stalk, the leaf blade otherwise glabrous and glossy on the upper surface. The color of the upper side of a mature leaf is Green Group 139A (Royal Horticultural Society Color Chart), and the color of the underside is Yellow-Green Group 146B. The leaf color is retained well through the winter.
Flowers: Not seen. Flowers are ornamentally insignificant in boxwood.
Fruits: Not produced so far. If produced, should not differ from the species.
The new and distinct variety has been named the WEE WILLIE.

1. A new and distinct winter-hardy cultivar of Buxus sinica var. insularis, possessing the following characteristics:
a semi dwarf evergreen shrub with decussate leaf arrangement, providing a dense, compact shrub with unique and pleasing texture for use in small landscapes, masses and low hedges; substantially as herein shown and described.
